(19) United States
(12) Patent Application Publication
(10) Pub. No.: US 2018/0296184 A1
(43) Pub. Date: Oct. 18, 2018

(54) ULTRASOUND DIAGNOSTIC APPARATUS AND ULTRASOUND PROBE

(71) Applicant: Konica Minolta, Inc., Tokyo (JP)

(72) Inventors: Tatsuya NAITO, Tokyo (JP); Shuhei Okuda, Tokyo (JP)

(21) Appl. No.: 15/947,312

(22) Filed: Apr. 6, 2018

(30) Foreign Application Priority Data
Apr. 12, 2017 (JP) 2017-079100

(51) Int. Cl. A61B 8/08; A61B 8/00

(52) U.S. Cl. CPC A61B 8/0841; A61B 8/54; A61B 8/4444; A61B 8/4483; A61B 8/5207; A61B 8/461; A61B 8/5246

(57) ABSTRACT

An ultrasound diagnostic apparatus includes: control section that switches a needle visibility priority mode in which ultrasound is not allowed to be transmitted from/received to transducer region including a central portion, but ultrasound is allowed to be transmitted from/received to transducer regions and excluding the central portion, and an image quality priority mode in which ultrasound is allowed to be transmitted from/received to transducer region; and an image generation section that generates needle visibility priority image data on the basis of reception signals in the needle visibility priority mode, and generates image quality priority image data on the basis of reception signals in the image quality priority mode.

### CROSS REFERENCE TO RELATED APPLICATIONS

The entire disclosure of Japanese Patent Application No. 2017-079100 filed on Apr. 12, 2017, is incorporated herein by reference in its entirety.

### BACKGROUND

### Technological Field

The present invention relates to an ultrasound diagnostic apparatus utilizing ultrasound, and an ultrasound probe of the ultrasound diagnostic apparatus.

### Description of Related Art

Ultrasound diagnostic apparatuses, which irradiate the inside of test objects with ultrasound and inspect the inside of the test objects by receiving and analyzing the reflected waves, have been widely used. Such ultrasound diagnostic apparatuses can examine test objects non-destructively and non-invasively, and thus are widely employed in various applications, such as medical diagnosis and inspection of the inside of architectural constructions.

In an ultrasound diagnostic apparatus, a plurality of acoustic elements (transducers) that convert voltage signals into ultrasonic vibration and vice versa are arranged in a predetermined direction (scanning direction), and the acoustic elements emit ultrasound upon application of driving voltage. Such an ultrasound diagnostic apparatus can acquire two-dimensional data in nearly real time by temporally changing (scanning) acoustic elements that detect voltage changes due to incidence of reflected ultrasound.

Further, an ultrasound diagnostic apparatus is used not only for image diagnosis, but also for biopsy in which a tissue of a subject, for example, is extracted. Specifically, in order to accurately puncture a site of interest, such as a tumor, real-time monitoring of the site of interest as well as a puncture needle is performed using an ultrasound diagnostic apparatus.

In some cases, however, real-time monitoring of a puncture needle becomes difficult when the needle does not advance in a planned puncture direction or the needle curves on the way to a site of interest due to effects of a position of a lesion and/or an insertion angle of the puncture needle. In such cases, accurate puncture becomes difficult.

Japanese Patent Application Laid-Open No. 2014-100556 (Patent Literature (PTL) 1), for example, discloses an ultrasound diagnostic apparatus that performs transmission/reception in a transmission waveform with a relatively low frequency, that performs image processing using a puncture needle enhanced image acquired using, of reception signals, the fundamental component of the frequency of the transmission waveform, and using a body tissue image acquired by changing scanning angles so as to generate a puncture needle extraction image in which only pixels corresponding to a puncture needle are extracted, and that generates an ultrasound image with a suitably presented puncture needle by superimoposing the puncture needle extraction image on a biological high-resolution image acquired by an imaging method and/or transmission/reception settings that enable good representation of a body tissue.

The technique disclosed in PTL 1, however, during acquisition of the puncture needle enhanced image, performs oblique scanning, in which the transmission/reception direction of ultrasonic vibration is a perpendicular direction to the longitudinal direction of the needle, and thus presupposes that an insertion angle of the needle is known in advance. Accordingly, in the technique disclosed in PTL 1, suitable monitoring of a puncture needle is difficult when an insertion angle of the needle differs from a planned angle due to curving of the needle on the way to a site of interest, for example.

### SUMMARY

An object of the present invention is to provide an ultrasound diagnostic apparatus that enables suitable monitoring of a puncture needle.

In order to achieve at least one of the abovementioned objects, an ultrasound diagnostic apparatus reflecting one aspect of the present invention includes: an ultrasound probe containing a transducer array in which a plurality of transducers are arranged in each of a long-axis direction and a short-axis direction; a control section that switches a first state in which ultrasound is not allowed to be transmitted from/received to at least a central transducer region including the transducers located in a central portion in the short-axis direction, but ultrasound is allowed to be transmitted from/received to a transducer region excluding the central transducer region, and a second state in which ultrasound is allowed to be transmitted from/received to at least the central transducer region in the short-axis direction; a reception processing section that receives, from the ultrasound probe, a first reception signal obtained through receiving of ultrasound, by the ultrasound probe, which has been transmitted from the ultrasound probe in the first state and reflected by a subject, and that receives, from the ultrasound probe, a second reception signal obtained through receiving of ultrasound, by the ultrasound probe, which has been transmitted from the ultrasound probe in the second state and reflected by the subject; and an image generation section that generates, on the basis of the first reception signal, first ultrasound image data which is image data including at least a puncture needle inserted into the subject, and that generates, on the basis of the second reception signal, second ultrasound image data which is image data including at least a body tissue of the subject.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 illustrates a configuration of an ultrasound diagnostic apparatus;

FIG. 2 is a block diagram illustrating an internal configuration of the ultrasound diagnostic apparatus;

FIG. 3 illustrates a transducer array of an ultrasound probe;

FIG. 4A illustrates a sectional structure of the transducer array in the short-axis direction, in which all switching elements 230a to 230c are in on-state;

FIG. 4B illustrates a sectional structure of the transducer array in the short-axis direction, in which switching elements 230a and 230c are in on-state;

FIG. 5 shows a relationship between on/off-state of switching elements 230a to 230c and transmission direction of ultrasound;

FIG. 6 is a diagram for illustrating puncture needle extraction operations and puncture needle pixel synthesis operations; and

FIG. 7 is a flow chart showing example operations of the ultrasound diagnostic apparatus when a puncture needle is used.

### DETAILED DESCRIPTION OF EMBODIMENTS

Hereinafter, one or more embodiments of the present invention will be described with reference to the drawings. However, the scope of the invention is not limited to the disclosed embodiments.

<Configuration of Ultrasound Diagnostic Apparatus>

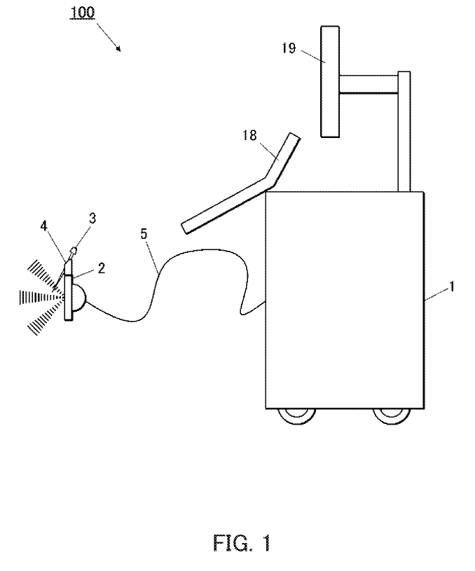

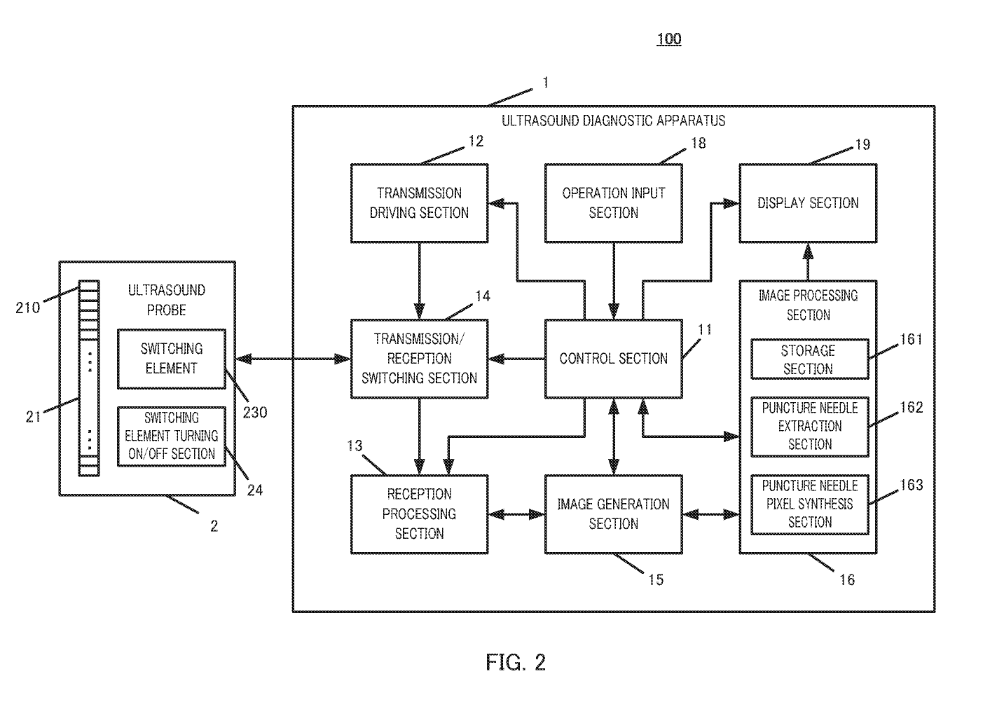

FIG. 1 illustrates the entire configuration of ultrasound diagnostic apparatus 100. FIG. 2 is a block diagram illustrating the internal configuration of ultrasound diagnostic apparatus 100.

As illustrated in FIG. 1, ultrasound diagnostic apparatus 100 includes ultrasound diagnostic apparatus body 1, ultrasound probe 2 connected with ultrasound diagnostic apparatus body 1 through cable 5, puncture needle 3, and holder 4 mounted on ultrasound probe 2.

Puncture needle 3 has a hollow long-needle shape, for example, and is inserted into a subject at a predetermined angle (hereinafter, referred to as insertion angle). Depending on purposes of puncture, the gauge, the length, and/or the tip shape of puncture needle 3 may be changed appropriately.

Holder 4 is mounted on the side portion of ultrasound probe 2, for example, and holds puncture needle 3 in a predetermined orientation (direction). By such a configuration, puncture needle 3 is intended to be located within a predetermined range including the ultrasound transmission/reception surface of ultrasound probe 2 when puncture needle 3 is inserted into a subject. The phrase “predetermined range” herein refers to a moving range when the ultrasound transmission/reception surface of ultrasound probe 2 is moved in the short-axis direction by a predetermined distance. Such a predetermined range is a range in which ultrasound image data can be generated (imaged) by ultrasound diagnostic apparatus 100. The holding direction of puncture needle 3 by holder 4 can be changed appropriately. Instead of holder 4, a guide portion for holding puncture needle 3 toward the insertion direction may be provided in ultrasound probe 2.

In ultrasound diagnostic apparatus body 1, operation input section 18 and display section 19 are provided. In addition, as illustrated in FIG. 2, ultrasound diagnostic apparatus body 1 includes control section 11, transmission driving section 12, reception processing section 13, transmission/reception switching section 14, image generation section 15, and image processing section 16.

Control section 11 performs control of the above-mentioned components other than control section 11. Control section 11 includes, for example, a central processing unit (CPU), a recording medium, such as read only memory (ROM) that stores a control program, and working memory, such as random access memory (RAM). The functions of control section 11 are realized by a CPU through reading and executing of a control program from ROM. A control program may be stored in a hard disk drive (HDD), a solid state drive (SSD), or an auxiliary storage apparatus, such as flash memory.

Transmission driving section 12 generates a pulse signal (transmission signal), which is to be supplied to ultrasound probe 2, according to a control signal input from control section 11, and outputs the pulse signal to transmission/reception switching section 14. Transmission driving section 12 includes a clock generator, a pulse width setting section, a pulse generator circuit, and a delay circuit, for example. The clock generator is a circuit that generates a clock signal, which determines transmission timing and/or transmission frequency of a pulse signal. The pulse width setting section sets a waveform (shape) of a transmission pulse output from a pulse generator circuit, a voltage amplitude, and a pulse width. The pulse generator circuit generates a transmission pulse on the basis of the settings by the pulse width setting section, and outputs the transmission pulse to wiring paths different for every transducer 210 of ultrasound probe 2. The delay circuit counts a clock signal output from the clock generator, and causes the pulse generator circuit to generate a transmission pulse after a preset delay time passes and to output the transmission pulse to each wiring path.

Reception processing section 13 acquires, from transmission/reception switching section 14, a reception signal input from ultrasound probe 2. Reception processing section 13 includes an amplifier, an A/D converter circuit, and a beamformer circuit, for example. The amplifier is a circuit that amplifies a reception signal corresponding to ultrasound received from each transducer 210 of ultrasound probe 2 by a preset amplification factor. The A/D converter circuit is a circuit that converts an amplified reception signal into digital data at a predetermined sampling frequency. The beamformer circuit is a circuit that generates sound ray data by giving delay time to A/D-converted reception signals in every wiring path corresponding to each transducer 210 to align the temporal phases, and by summing them (delay and sum).

Transmission/reception switching section 14 performs, on the basis of control by control section 11, transmission/reception switching operations for outputting a transmission signal from transmission driving section 12 to ultrasound probe 2 when ultrasound is emitted (transmitted) from ultrasound probe 2, and for inputting a reception signal into reception processing section 13 when a reception signal concerning ultrasound that has been received by ultrasound probe 2 is input.

Transmission/reception processing of ultrasound diagnostic apparatus 100 is performed by the above-mentioned transmission driving section 12, reception processing section 13, and transmission/reception switching section 14.

Image generation section 15 generates diagnostic image data on the basis of reception signals of ultrasound. Image generation section 15 detects sound ray data input from reception processing section 13 (envelope detection) to acquire signals, and performs logarithmic amplification, filtering (low-pass or smoothing, for example), and/or enhancement processing, for example, as appropriate. Image generation section 15 generates, as diagnostic ultrasound image data, diagnostic image data (in every frame) concerning B-mode presentation (brightness-mode presentation) showing the inside of a subject as brightness signals corresponding to the signal intensity of reception signals in a cross-section, which includes the ultrasound transmission/reception direction (depth direction of a subject) and the scanning direction (second direction) of ultrasound transmitted by ultrasound probe 2. Image generation section 15 may be configured to perform dynamic range adjustment and/or gamma correction, for example, during displaying of ultrasound image data.

Image generation section 15 may include a CPU and/or RAM dedicated for ultrasound image generation. Alternatively, image generation section 15 may be composed of hardware configuration concerning image data generation formed on a substrate (application-specific integrated circuit: ASIC, for example) or a field-programmable gate array (FPGA). Further, image generation section 15 may be configured so that processing concerning image data generation is performed by the above-mentioned CPU and RAM of control section 11.

Image processing section 16, as illustrated in FIG. 2, includes storage section 161, puncture needle extraction section 162, and puncture needle pixel synthesis section 163. Storage section 161 stores diagnostic image data that is processed in image generation section 15 and used for real-time presentation (or quasi real-time presentation) of a predetermined number of latest frames. Storage section 161 may be volatile memory, such as dynamic random access memory (DRAM) or various types of nonvolatile memory that enables high-speed rewriting.

Diagnostic image data stored in storage section 161 is read according to control by control section 11, and for example, transmitted to display section 19 or output outside ultrasound diagnostic apparatus 100 through communication section (not shown).

Puncture needle extraction section 162 extracts pixels corresponding to puncture needle 3 within ultrasound image data generated by image generation section 15. Extracted pixels are preferably a plurality of pixels corresponding to the entire puncture needle 3 within ultrasound image data, but may be pixels within a predetermined range including only the tip portion of puncture needle 3 depending on detection accuracy and/or resolution of image data, for example. Processing in puncture needle extraction section 162 may be performed by a dedicated CPU and RAM, performed by a CPU and RAM of image processing section 16, or performed by a CPU and RAM of control section 11. The details of puncture needle extraction operations by puncture needle extraction section 162 will be described hereinafter.

Puncture needle pixel synthesis section 163 performs puncture needle pixel synthesis operations for combining pixels corresponding to puncture needle 3 extracted by puncture needle extraction section 162 with other ultrasound image data. The details of puncture needle pixel synthesis operations by puncture needle pixel synthesis section 163 will be described hereinafter.

Operation input section 18 is composed of a push button, a switch, a keyboard, a mouse, a track ball, touch panel, or combinations thereof. Operation input section 18 converts input operations by a user into operation signals and outputs the operation signals to control section 11.

Display section 19 is a display device, such as a liquid crystal display (LCD), an organic electroluminescent (EL) display, an inorganic EL display, a plasma display, or a cathode ray tube (CRT), for example. Display section 19 generates a driving signal of a display screen (each display pixel) according to a control signal output from control section 11 and/or image data generated by image processing section 16, and shows, on the display screen, a menu concerning ultrasound diagnosis, the status, and measured data based on received ultrasound.

Operation input section 18 and/or display section 19 may be provided integrally with a housing for ultrasound diagnostic apparatus body 1, or may be externally attached through various cables.

On the basis of control by control section 11, ultrasound probe 2 oscillates ultrasound at about 1 to 30 MHz, for example, transmits the ultrasound from its ultrasound transmission/reception surface to a subject, receives, of the transmitted ultrasound, the reflected wave (echo) by the subject, and converts the reflected wave into a reception signal. Ultrasound probe 2 includes transducer array 21, which is an array of a plurality of transducers 210 that transmit/receive ultrasound, a plurality of switching elements 230, and switching element turning on/off section 24.

In ultrasound diagnostic apparatus 100 of an embodiment of the present invention, ultrasound probe 2 is described as a probe that transmits ultrasound from the outside (surface) of a subject and receives the reflected wave. The present invention, however, is not limited to such a probe. Ultrasound probe 2, for example, may have the size or the shape that can be used by inserting inside a subject, such as a digestive organ, a blood vessel, or a body cavity.

Transducer array 21 is an array of a plurality of transducers 210 equipped with piezoelectric elements in which electrodes are arranged on both ends of piezoelectric materials.

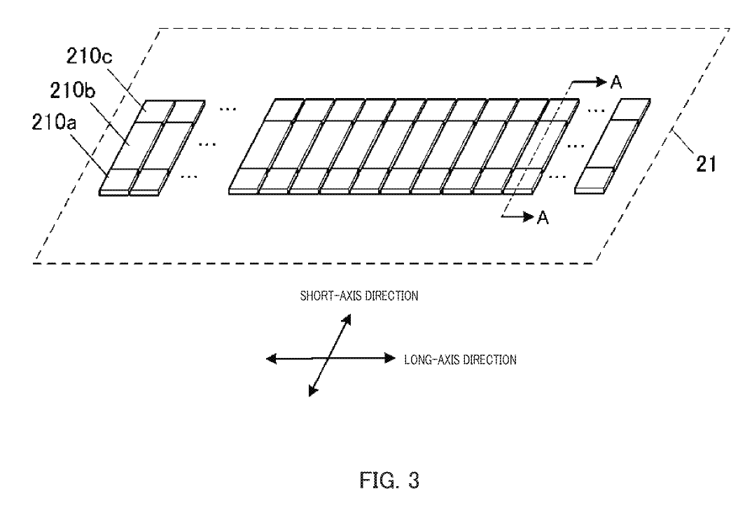

FIG. 3 illustrates transducer array 21 of ultrasound probe 2. As illustrated in FIG. 3, transducer array 21 is composed of a plurality of transducers 210 arranged as a matrix in a two-dimensional plane defined by the scanning direction and the width direction orthogonal to the scanning direction. The two-dimensional plane, in which a plurality of transducers 210 are arranged, is not necessarily a plane. Since the number of transducers 210 arranged in the scanning direction is generally larger than the number of transducers 210 arranged in the width direction, the scanning direction is regarded as the long-axis direction and the width direction as the short-axis direction.

In ultrasound diagnostic apparatus 100 of the embodiment, as illustrated in FIG. 3, three transducer regions 210a, 210b, and 210c are formed in this order in the short-axis direction of transducer array 21. Hereinafter, a group of three transducer regions 210a to 210c arranged in the short-axis direction are referred to as a transducer column. A plurality of such transducer columns are arranged in the scanning direction. Transducer regions 210a and 210c are regions including both end portions in the short-axis direction of transducer array 21, whereas transducer region 210b is a region including a central portion in the short-axis direction of transducer array 21.

A transmission signal is input into each transducer included in the respective transducer regions 210a to 210c from transmission/reception switching section 14 of ultrasound diagnostic apparatus body 1. In ultrasound diagnostic apparatus 100 of the embodiment, a transmission signal is input into transducers 210 in the same transducer region simultaneously. Transmission/reception signal lines 211 are connected to the respective transducers 210 included in the respective transducer regions 210a to 210c. For the sake of simplicity, transmission/reception signal lines 211 connected to transducers 210 of transducer regions 210a to 210c are each illustrated and described collectively as transmission/reception signal lines 211a to 211c in the description hereinafter.

When a transmission signal (voltage pulse) is input into the respective transducers 210 of transducer regions 210a to 210c through transmission/reception signal lines 211a to 211c, ultrasound is transmitted from transducers 210 in a region into which the transmission signal has been input. The transmitted ultrasound is emitted to a position and in a direction corresponding to the position and direction of transducers 210 included in a predetermined number of transducer columns to which a voltage pulse is supplied, as well as to the convergence direction and the magnitude of lag in timing (delay) of the transmitted ultrasound. The number of transducer columns to which a transmission signal is supplied in the scanning direction may be one or more.

When ultrasound reflected by a subject enters transducer regions 210a to 210c, the resulting sound pressure changes the thickness of a piezoelectric material of each transducer 210 (vibration), thereby generating charges corresponding to the change. Each transducer region 210a to 210c outputs, as a reception signal, an electrical signal corresponding to the charges in every transducer region to transmission/reception switching section 14 of ultrasound diagnostic apparatus body 1 through transmission/reception signal lines 211a to 211c.

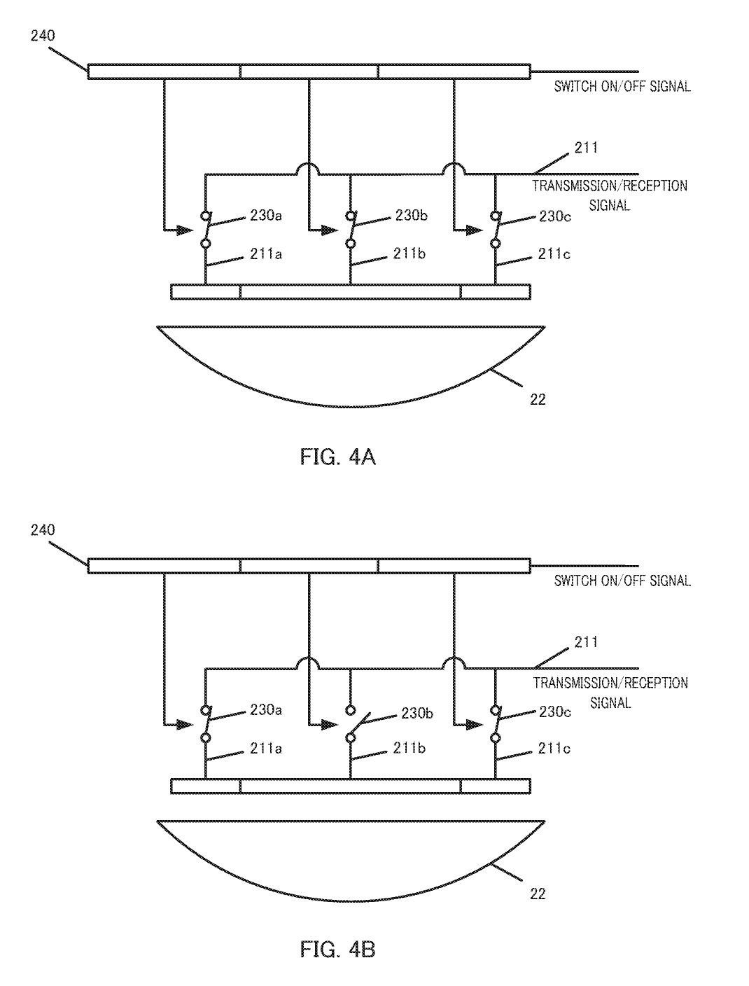

Transmission/reception signal lines 211a to 211c are connected with switching element turning on/off section 24 through the respective switching elements 230a to 230c. Switching element turning on/off section 24 includes register 240 (see FIG. 4A and FIG. 4B), and switching element on/off signals stored in register 240 in advance are output to switching elements 230a to 230c in every predetermined cycle.

Switching elements 230a to 230c are turned on/off on the basis of input switching element on/off signals. Hereinafter, turning on/off operations of switching elements 230a to 230c by switching element turning on/off section 24 is referred to as switching element turning on/off operations. The details of the switching element turning on/off operations will be described hereinafter. Meanwhile, switching element on/off signals are transmitted in series and input into register 240, and enables control of operations of the respective switching elements 230a to 230c in parallel, thereby reducing the number of signal lines between control section 11 and register 240.

As switching elements 230a to 230c, a field effect transistor (FET), for example, is used. In the present invention, switching elements 230a to 230c are not limited to a FET, but it is preferable to use elements in which power consumption and withstand voltage performance, for example, are taken into account.

When any of switching elements 230a to 230c is turned off by a switching element on/off signal, a transmission signal output from ultrasound diagnostic apparatus body 1 is not input into transducer region 210a to 210c corresponding to the switching element that has been turned off, and consequently, a reception signal output from the corresponding transducer region 210a to 210c is not input into ultrasound diagnostic apparatus body 1. When switching element 230 is turned on, a transmission signal output from ultrasound diagnostic apparatus body 1 is input into transducer 210, and thus a reception signal output by transducer 210 is input into ultrasound diagnostic apparatus body 1.

Cable 5 includes, in the both ends, a connector (not shown) to ultrasound diagnostic apparatus body 1 and a connector (not shown) to ultrasound probe 2. Ultrasound probe 2 is configured to be detachable from ultrasound diagnostic apparatus body 1 by cable 5. Cable 5 may be formed integrally with ultrasound probe 2.

<Details of Switching Element Turning on/Off Operations>

In the following, switching element turning on/off operations performed by switching element turning on/off section 24 of ultrasound probe 2 will be described in detail. Switching element turning on/off operations by switching element turning on/off section 24 are performed on the basis of control by control section 11 of ultrasound diagnostic apparatus body 1, for example.

Switching element turning on/off section 24 performs turning on/off operations of switching elements 230a to 230c in a preset cycle. More specifically, switching element turning on/off section 24, in every predetermined cycle, outputs a switching element on/off signal to switching element 230b without outputting a switching element on/off signal to switching elements 230a and 230c. The predetermined cycle herein refers to each generation frame of ultrasound image data, for example.

In other words, switching elements 230a and 230c are always in on-state whereas switching element 230b repeats on/off-state every predetermined cycle. Accordingly, transducers 210 in transducer regions 210a and 210c keep transmitting ultrasound while transducers 210 in transducer region 210b repeat transmission of ultrasound and its suspension in every predetermined cycle. FIG. 4A illustrates a sectional structure of transducer array 21 in the short-axis direction, in which all switching elements 230a to 230c are in on-state. FIG. 4B illustrates a sectional structure of transducer array 21 in the short-axis direction, in which switching elements 230a and 230c are in on-state. FIGS. 4A and 4B are A-A cross-section in FIG. 3.

As illustrated in FIGS. 4A and 4B, acoustic lens 22 of a convex lens shape is provided in ultrasound probe 2 so as to cover ultrasound emission directions of three transducer regions 210a, 210b, and 210c included in one transducer column. Acoustic lens 22 refracts the transmission direction of ultrasound transmitted from transducer regions 210a to 210c and the reception direction of ultrasound (echo) incident on transducer regions 210a to 210c, and thus the transmission/reception width is converged in the short-axis direction of ultrasound probe 2. As a material for acoustic lens 22, a silicone is used, for example. Alternatively, other materials may be selected appropriately as a material for acoustic lens 22 depending on a desired refractive index of ultrasound.

Acoustic lens 22 and register 240 illustrated in FIGS. 4A and 4B are omitted in FIG. 3.

As illustrated in FIG. 4A, when all switching elements 230a to 230c are in on-state, a transmission signal is input into all transducers 210 in transducer regions 210a to 210c, and ultrasound is transmitted from all transducers 210 in transducer regions 210a to 210c. Meanwhile, as illustrated in FIG. 4B, when switching elements 230a and 230c are in on-state, a transmission signal is input into transducers 210 in transducer regions 210a and 210c, but not into transducers 210 in transducer region 210b. Accordingly, when switching elements 230a and 230c are in on-state, ultrasound is transmitted from transducers 210 in transducer regions 210a and 210c, but not from transducers 210 in transducer region 210b. In the description hereinafter, a state in which ultrasound is transmitted from all transducers 210 in transducer regions 210a to 210c as illustrated in FIG. 4A is referred to as “image quality priority mode,” whereas a state in which ultrasound is transmitted from transducers 210 in transducer regions 210a and 210c as illustrated in FIG. 4B is referred to as “needle visibility priority mode.” The image quality priority mode is an example of the second state of the present invention, whereas the needle visibility priority mode is an example of the first state of the present invention. Meanwhile, a reception signal generated by receiving of ultrasound, by transducers 210, which has been transmitted in the image quality priority mode and reflected by a subject is an example of the second reception signal, whereas a reception signal generated by receiving of ultrasound, by transducers 210, which has been transmitted in the needle visibility priority mode and reflected by the subject is an example of the first reception signal.

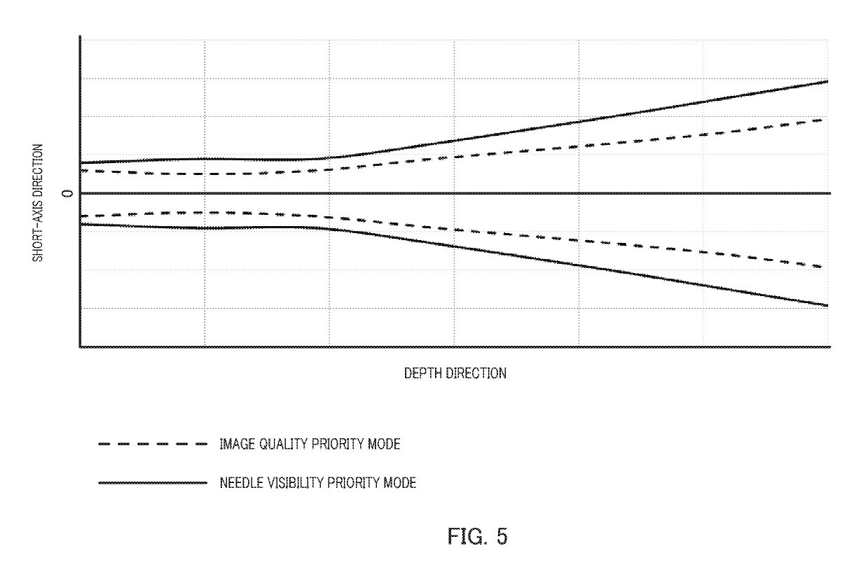

FIG. 5 shows a relationship between on/off-state of switching elements 230a to 230c and transmission direction of ultrasound. In FIG. 5, the horizontal axis corresponds to the depth in the depth direction from transducers 210 in transducer regions 210a to 210c, and the vertical axis corresponds to the distance from the central portion in the short-axis direction. In other words, the dotted lines and the solid lines of FIG. 5 show the transmission direction of ultrasound transmitted from transducers 210 located near the center in the left end of FIG. 5. This means that FIG. 5 illustrates the extent of transmission ultrasound (ultrasound beams) in the short-axis direction of ultrasound probe 2. The dotted lines of FIG. 5 correspond to the extent of transmission ultrasound in the image quality priority mode, whereas the solid lines of FIG. 5 correspond to the extent of transmission ultrasound in the needle visibility priority mode. In ultrasound diagnostic apparatus 100 of the embodiment, as shown in FIG. 5, ultrasound beams are line-symmetric about a direction passing through the central portion in the short-axis direction (corresponding to “0” in the vertical axis of FIG. 5) and being perpendicular to the short-axis direction (ultrasound transmission/reception direction).

In the Image quality priority mode, since ultrasound is transmitted from all transducers 210 in transducer regions 210a to 210c, the ultrasound beam is relatively restricted in its beam width as shown in FIG. 5. Since the ultrasound beam having a relatively restricted beam width is used in the image quality priority mode, ultrasound image data having relatively reduced artifacts and good image quality is generated when image generation section 15 generates such image data by using the second reception signal, which is a reception signal in the image quality priority mode.

Meanwhile, in the needle visibility priority mode, since ultrasound is transmitted from transducers 210 in transducer regions 210a and 210c, which are both end portions in the short-axis direction sandwiching the central portion, more side lobes are generated by intentionally caused interference. Accordingly, in the needle visibility priority mode, the ultrasound beam has a relatively wide beam width as shown in FIG. 5.

Since the ultrasound beam is relatively expanded in the needle visibility priority mode, even when puncture needle 3 curves and thus is located somewhat outside the wavefront of ultrasound, puncture needle 3 is readily located within the irradiation range of the ultrasound beam. Moreover, in the needle visibility priority mode, since the ultrasound beam is relatively expanded, even a small needle is readily found. Accordingly, the visibility of puncture needle 3 is enhanced in ultrasound image data generated by image generation section 15 by using the first reception signal, which is a reception signal in the needle visibility priority mode.

Although a case in which ultrasound is transmitted from transducers 210 in transducer regions 210a to 210c is explained in the above description of the switching element turning on/off operations, the same also applies to a case in which ultrasound reflected by a subject is received by transducers 210 in transducer regions 210a to 210c.

In the needle visibility priority mode, since transducers 210 in transducer region 210b are not used, the transmission intensity of ultrasound lowers relative to the image quality priority mode. Correspondingly, the reception intensity of ultrasound lowers. When image generation section 15 generates ultrasound image data on the basis of the first reception signal generated by transducers 210 in the needle visibility priority mode, clear ultrasound image data can be generated by multiplying the reception intensity by a coefficient corresponding to its lowering so as to harmonize (normalize) brightness distribution (gain). Alternatively, image generation section 15 may increase the reception intensity by changing (increasing) a voltage amplitude of a rectangular-wave pulse concerning transmission of ultrasound.

Since a S/N ratio lowers significantly due to lowering in transmission/reception intensity of ultrasound in the needle visibility priority mode, it is preferable to preset the width of transducer regions 210a to 210c and/or a voltage amplitude, for example, so as to have a S/N ratio (reception intensity) that ensures detection of puncture needle 3.

<Details of Puncture Needle Extraction Operations and Puncture Needle Pixel Synthesis Operations>

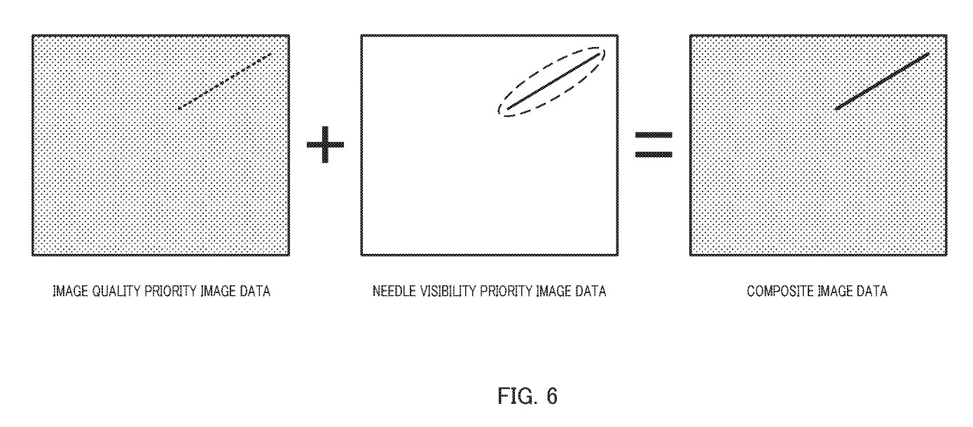

Next, puncture needle extraction operations performed by puncture needle extraction section 162 of image processing section 16 and puncture needle pixel synthesis operations by puncture needle pixel synthesis section 163 will be described in detail. FIG. 6 is a diagram for illustrating puncture needle extraction operations and puncture needle pixel synthesis operations.

As in the foregoing, the image quality priority mode and the needle visibility priority mode are switched in every predetermined cycle through switching element turning on/off operations by switching element turning on/off section 24. Image generation section 15 generates image quality priority image data, which is an ultrasound image with relatively good image quality of a body tissue, by using the second reception signal in the image quality priority mode Image generation section 15 also generates needle visibility priority image data, which is an ultrasound image with relatively excellent visibility of puncture needle 3, by using the first reception signal in the needle visibility priority mode. The image quality priority image data is an example of the second ultrasound image data of the present invention, whereas the needle visibility priority image data is an example of the first ultrasound image data of the present invention.

Puncture needle extraction section 162 extracts pixels corresponding to puncture needle 3 by using the image quality priority image data and the needle visibility priority image data. Specifically, puncture needle extraction section 162 extracts pixels corresponding to puncture needle 3 according to the following method. In other words, puncture needle extraction section 162 compares brightness values of the image quality priority image data and the needle visibility priority image data in every spatially corresponding position, and regards a brightness value of the needle visibility priority image data as a pixel value corresponding to puncture needle 3 in a position where the brightness value of the needle visibility priority image data is larger than that of the image quality priority image data. Through this process, only pixels corresponding to puncture needle 3 can be extracted.

In a position where a brightness value of the needle visibility priority image data is larger than that of the image quality priority image data, puncture needle extraction section 162 may extract pixels corresponding to puncture needle 3 by the following method, rather than simply regards the brightness value of the needle visibility propriety image data as a pixel value corresponding to puncture needle 3. In other words, in a position where a brightness value of the needle visibility priority image data is larger, a brightness value of the image quality priority image data and a brightness value of the needle visibility priority image data may undergo addition/subtraction operations or averaging, and the resulting value may be regarded as a pixel value corresponding to puncture needle 3.

As illustrated in FIG. 6, once puncture needle extraction section 162 extracts pixels corresponding to puncture needle 3, puncture needle pixel synthesis section 163 combines pixel values in positions corresponding to puncture needle 3 with the image quality priority image data. Through this process, ultrasound image data (hereinafter, referred to as composite image data) having excellent visibility of puncture needle 3 and relatively good image quality can be generated.

When puncture needle extraction section 162 performs puncture needle extraction operations, brightness values of the image quality priority image data and/or the needle visibility priority image data may be adjusted so that composite image data generated by puncture needle pixel synthesis section 163 becomes clear image data. A harder substance than a subject, such as puncture needle 3, has an extremely high acoustic impedance compared with a biological material, such as a subject. Accordingly, puncture needle 3 tends to be imaged at a higher brightness than a biological material in ultrasound image data. Therefore, by lowering brightness values of the pixels corresponding to puncture needle 3 in the needle visibility priority image data so as to correspond to those of the image quality priority image data, clearer composite image data can be generated.

<Example Operations of Ultrasound Diagnostic Apparatus>

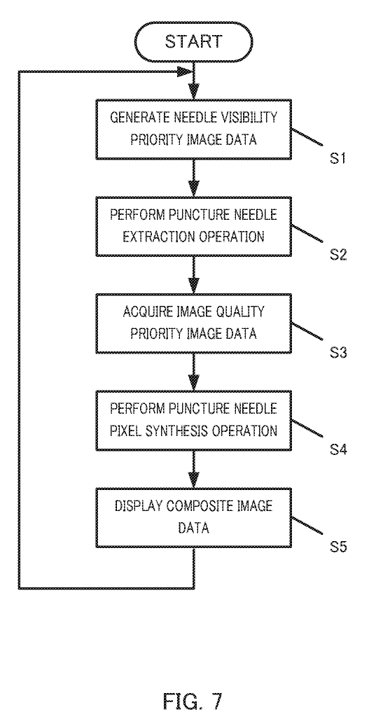

In the following, example operations of ultrasound diagnostic apparatus 100 when puncture needle 3 is used will be described. FIG. 7 is a flow chart showing example operations of ultrasound diagnostic apparatus 100 when puncture needle 3 is used.

In step S1, image generation section 15 generates needle visibility image data. Specifically, as in the foregoing, switching element turning on/off section 24 of ultrasound probe 2 switches to the needle visibility priority mode in which ultrasound is allowed to be transmitted from transducers 210 in transducer regions 210a and 210c by turning off switching element 230b such that a transmission signal is input into transducers 210 in transducer regions 210a and 210c, but not into transducers 210 in transducer region 210b Image generation section 15 then generates needle visibility priority image data by using a reception signal (first reception signal) received by transducers 210 in transducer regions 210a and 210c.

In step S2, puncture needle extraction section 162 of image processing section 16 performs puncture needle extraction operations for extracting pixels corresponding to puncture needle 3 by using the needle visibility priority image data generated in step S1.

In step S3, image generation section 15 generates image quality priority image data. Specifically, as in the foregoing, switching element turning on/off section 24 of ultrasound probe 2 switches to the image quality priority mode in which ultrasound is transmitted from all transducers 210 in transducer regions 210a to 210c by turning on switching element 230b such that a transmission signal is input into all transducers 201 in transducer regions 210a to 210c. Image generation section 15 then generates image quality priority image data by using a reception signal (second reception signal) received by transducers 210 in transducer regions 210a to 210c.

In step S4, puncture needle pixel synthesis section 163 of image processing section 16 generates composite image data with excellent visibility of puncture needle 3 and relatively good image quality by combining pixels corresponding to puncture needle 3 extracted in step S2 with image quality priority image data generated in step S3.

In step S5, display section 19 shows the composite image data generated in step S4.

In the flow chart shown in FIG. 7, two cycles of, in the above-mentioned predetermined cycles, operations of ultrasound diagnostic apparatus 100 are shown. In other words, in FIG. 7, step S1 and S2 correspond to one cycle of operations, and step S3 and step S4 also correspond to one cycle of operations. The operation of step S5 may be performed in either the cycles.

For example, when a predetermined cycle corresponds to a generation frame of ultrasound image data, since switching element turning on/off section 24 of ultrasound probe 2 outputs a switching element on/off signal to switching elements 230a to 230c in every frame, image generation section 15 alternately generates needle visibility priority image data and image quality priority image data in every frame. In this case, step S1 and step S2 correspond to operations of one frame, and step S3 and step S4 correspond to operations of another frame. Accordingly, in this case, a frame rate of composite image data shown by display section 19 in step S5 is a half that of normal operations (when puncture needle 3 is not used).

Once step S5 ends, processing of ultrasound diagnostic apparatus 100 returns to step S1 and moves to a subsequent cycle of generation operations of needle visibility priority image data.

FIG. 7 shows example operations of ultrasound diagnostic apparatus 100 when puncture needle 3 is used. When puncture needle 3 is not used, however, ultrasound diagnostic apparatus 100 does not turn on/off switching elements 230a to 230c in every predetermined cycle, but rather keeps generating ultrasound image data in the image quality priority mode.

Whether puncture needle 3 is used or not may be determined according to the following method. For example, when a user inputs the use of puncture needle 3 into ultrasound diagnostic apparatus 100 through operation input section 18, ultrasound diagnostic apparatus 100 determines that puncture needle 3 is used. Alternatively, when puncture needle extraction section 162 attempts puncture needle extraction operations for all frame image data of image quality priority image data generated by image generation section 15 in the image quality priority mode, and pixels corresponding to puncture needle 3 are extracted, puncture needle 3 is determined to be used. Once puncture needle 3 is determined to be used, ultrasound diagnostic apparatus 100 may move to the operations of the flow chart in FIG. 7.

As described above, ultrasound diagnostic apparatus 100 of the embodiment includes: ultrasound probe 2 containing a transducer array in which a plurality of transducers are arranged in each of a long-axis direction and a short-axis direction; control section 12 that switches a needle visibility priority mode in which ultrasound is not allowed to be transmitted from/received to at least transducer region 210b including the transducers located in the central portion in the short-axis direction, but ultrasound is allowed to be transmitted from/received to transducer regions 210a and 210c excluding transducer region 201b, and an image quality priority mode in which ultrasound is allowed to be transmitted from/received to at least transducer region 210b; reception processing section 13 that receives, from ultrasound probe 2, a first reception signal obtained through receiving of ultrasound, by ultrasound probe 2, which has been transmitted from ultrasound probe 2 in the needle visibility priority mode and reflected by a subject, and that receives, from ultrasound probe 2, a second reception signal obtained through receiving of ultrasound, by ultrasound probe 2, which has been transmitted from ultrasound probe 2 in the image quality priority mode and reflected by the subject; and image generation section 15 that generates, on the basis of the first reception signal, needle visibility priority image data including at least a puncture needle inserted into the subject, and generates, on the basis of the second reception signal, image quality priority image data including at least a body tissue of the subject.

By the above configuration, ultrasound diagnostic apparatus 100, in the needle visibility priority mode, can generate needle visibility priority image data with enhanced visibility of puncture needle 3 on the basis of a reception signal obtained by widening a beam width of the ultrasound beam in the short-axis direction relative to the image quality priority mode. Accordingly, puncture needle 3 can be monitored suitably.

In ultrasound diagnostic apparatus 100 of the embodiment, ultrasound probe 2 includes switching elements 230a to 230c that allow and block transmission/reception of ultrasound from/to transducers 210 in every transducer region 210a to 210c, and switching element turning on/off section 24 that turns on/off switching elements 230a to 230c in every predetermined cycle so as to allow alternate transition to the needle visibility priority mode and to the image quality priority mode when puncture needle 3 is inserted.

By the above configuration, since ultrasound beams irradiated by ultrasound diagnostic apparatus 100 are relatively expanded, puncture needle 3 is readily found even when an insertion angle differs from a preset angle due to curving of puncture needle 3 inserted into a subject on the way to a site of interest, for example. Moreover, even when a small puncture needle is used, puncture needle 3 is readily found.

Further, in ultrasound diagnostic apparatus 100 of the embodiment, switching turning on/off section 24 turns on/off switching elements 230a to 230c in the needle visibility priority mode such that ultrasound is allowed to be transmitted from/received to two or more line-symmetric regions about a direction passing through the central portion in the short-axis direction and being perpendicular to the short-axis direction.

By the above configuration, ultrasound diagnostic apparatus 100 can widen the ultrasound beam width without changing the aperture width. By widening the ultrasound beam width without changing the aperture width, puncture needle 3 can be monitored suitably without lowering in slice resolution of ultrasound beams.

In an ultrasound diagnostic apparatus, widening of the ultrasound beam width without changing the aperture width is preferable due to the following reasons. The slice resolution generally improves as the ultrasound beam width narrows. Accordingly, in some cases, the aperture width in the short-axis direction is designed to be narrow in the stage of designing a housing for an ultrasound probe. In the present invention, even when the aperture width is designed to be narrow, the beam width can be changed by switching channels, and thus both image quality and visibility of a needle can be achieved simultaneously.

<Modifications>

In the above-described embodiment, transducer array 21 includes three transducer regions 210a to 210c in the short-axis direction. The present invention, however, is not limited to this configuration. Three or more transducer regions may be provided in the short-axis direction. Since one of a plurality of transducer regions provided in the short-axis direction needs to include transducers 210 in the central portion in the short-axis direction, the number of transducer regions included in transducer array 21 is an odd number.

When three or more transducer regions are provided in the short-axis direction, in order to widen the beam width of ultrasound beams in the short-axis direction, ultrasound needs to be transmitted from/received to transducers in transducer regions excluding a transducer region including the central portion. In this case, transducer regions in which ultrasound is allowed to be transmitted from/received to are not necessarily regions including both end portions in the short-axis direction. Specifically, suppose that five transducer regions are provided in the short-axis direction and the third transducer region is a transducer region including the central portion, only transducers 210 in the first and the fourth transducer regions may be allowed to transmit/receive ultrasound, or only transducers 210 in the first and the fifth transducer regions may be allowed transmit/receive ultrasound, for example. Alternatively, only transducers 210 in the second and the fourth transducer regions may be allowed to transmit/receive ultrasound, or only transducers 210 in the second and the fifth transducer regions may be allowed to transmit/receive ultrasound. Further, transducers in a plurality of transducer regions, such as the first, the second, and the fourth regions, may be allowed to transmit/receive ultrasound. In this case, ultrasound beams transmitted from ultrasound probe 2 are preferably transmitted in a perpendicular direction to the ultrasound transmission/reception surface by adjusting the width of transducer regions in advance, for example.

In the above-described embodiment, as illustrated in FIGS. 4A and 4B, transducer region 210a and transducer region 210c are line-symmetric about a direction passing through the central portion in the short-axis direction and being perpendicular to the short-axis direction. The present invention, however, is not limited to this configuration. Transducer region 210a and transducer region 210c may not be arranged line-symmetric. When transducer region 210a and transducer region 210c are not arranged line-symmetric, ultrasound beams are not line-symmetric beams about the direction perpendicular to the short-axis direction as illustrated in FIG. 5. When ultrasound is transmitted from transducers 210 in transducer regions 210a and 210c, however, the beam width is widened compared with a case in which ultrasound is transmitted from all transducers 210 in transducer regions 210a to 210c. Accordingly, such a case is still preferable from a viewpoint of enhancing visibility of puncture needle 3.

### INDUSTRIAL APPLICABILITY

The present invention is suitable for an ultrasound diagnostic apparatus that can conduct ultrasound diagnosis using a puncture needle.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound diagnostic apparatus comprising:
an ultrasound probe containing a transducer array in which a plurality of transducers are arranged in each of a long-axis direction and a short-axis direction;
a controller that switches a first state in which ultrasound is not allowed to be transmitted from/received to at least a central transducer region including the transducers located in a central portion in the short-axis direction, but ultrasound is allowed to be transmitted from/received to a transducer region excluding the central transducer region, and a second state in which ultrasound is allowed to be transmitted from/received to at least the central transducer region in the short-axis direction;
a reception processor that receives, from the ultrasound probe, a first reception signal obtained through receiving of ultrasound, by the ultrasound probe, which has been transmitted from the ultrasound probe in the first state and reflected by a subject, and that receives, from the ultrasound probe, a second reception signal obtained through receiving of ultrasound, by the ultrasound probe, which has been transmitted from the ultrasound probe in the second state and reflected by the subject; and
an image generator that generates, on the basis of the first reception signal, first ultrasound image data which is image data including at least a puncture needle inserted into the subject, and that generates, on the basis of the second reception signal, second ultrasound image data which is image data including at least a body tissue of the subject.

2. The ultrasound diagnostic apparatus according to claim 1, further comprising an image processor that generates composite image data on the basis of the first ultrasound image data and the second ultrasound image data.

3. The ultrasound diagnostic apparatus according to claim 2, wherein the image processor extracts, on the basis of the first ultrasound image data, image data corresponding to the puncture needle inserted into the subject, and generates the composite image data on the basis of the second ultrasound image data and the image data corresponding to the puncture needle.

4. The ultrasound diagnostic apparatus according to claim 1, wherein the ultrasound probe includes:
one or more switches that allow and block transmission/reception of ultrasound in each of the transducer regions; and
a switch turning on/off part that turns on/off the one or more switches so as to allow transition to the first state and to the second state on the basis of control by the controller.

5. The ultrasound diagnostic apparatus according to claim 4, wherein the switch turning on/off part maintains the one or more switches corresponding to at least the central transducer region in on-state when the puncture needle is not inserted.

6. The ultrasound diagnostic apparatus according to claim 4, wherein the switch turning on/off part, in the first state, turns on/off the switches such that ultrasound is allowed to be transmitted from/received to two or more transducer regions located in both sides of the central transducer region among the transducer regions.

7. The ultrasound diagnostic apparatus according to claim 4, wherein the switch turning on/off part, in the first state, turns on/off the switches such that ultrasound is allowed to be transmitted from/received to two or more transducer regions which are line-symmetric about a direction passing through the central portion in the short-axis direction and being perpendicular to the short-axis direction.

8. An ultrasound probe comprising:
a transducer array in which a plurality of transducers are arranged in each of a long-axis direction and a short-axis direction;
one or more switches that allow and block transmission/reception of ultrasound from/to at least a central transducer region including the transducers located in a central portion in the short-axis direction; and
a switch turning on/off part that turns on/off the switches so as to allow transition to a first state in which ultrasound is not allowed to be transmitted from/received to at least the central transducer region including the transducers located in the central portion in the short-axis direction, but ultrasound is allowed to be transmitted from/received to a transducer region excluding the central transducer region, and a second state in which ultrasound is allowed to be transmitted from/received to at least the central transducer region in the short-axis direction.
